(19) United States
(12) Patent Application Publication
(10) Pub. No.: US 2024/0122879 A1
(43) Pub. Date: Apr. 18, 2024

(54) COMPOSITIONS AND METHODS FOR TREATING OCULAR CHEMICAL BURNS

(71) Applicant: OHIO STATE INNOVATION FOUNDATION, Columbus, OH (US)

(72) Inventors: Hua Zhu, Columbus, OH (US); Peng Chen, Columbus, OH (US)

(21) Appl. No.: 18/375,697

(22) Filed: Oct. 2, 2023

Related U.S. Application Data
(60) Provisional application No. 63/378,112, filed on Oct. 3, 2022.

(51) Int. Cl. A61K 31/145; A61P 27/02

(52) U.S. Cl. CPC A61K 31/145; A61P 27/02

(57) ABSTRACT

Disclosed herein are methods for treating ocular chemical burns in a subject that involves administering to the cornea of a subject in need thereof a composition comprising a pyroptosis inhibitor, such as disulfiram.

### CROSS REFERENCE TO RELATED APPLICATIONS

This application claims the benefit of U.S. Provisional Application No. 63/378,112 filed on Oct. 3, 2022, which is incorporated herein by reference in their entireties.

### STATEMENT REGARDING FEDERALLY SPONSORED RESEARCH OR DEVELOPMENT

This invention was made with Government Support under Grant Nos. EY030621 and EY032583 awarded by the National Institutes of Health. The Government has certain rights in the invention.

### BACKGROUND

Ocular burns account for 3-4% of occupational injuries and 7-18% of ocular traumas in the United States, with the majority of these being chemical injuries (Bunker, et al. J Burn Care Res. 2014 35(3):261-8). Chemical burns have been reported to cause up to 36,000 visits to emergency departments annually (Bates, et al. Ocular Burns. 2020), and while accidents leading to ocular burns occur at all ages, individuals between 18 and 64 years of age are most commonly affected (Haring, et al. JAMA ophthalmology 2016 134:1119-1124).

Chemical burns to the eye are usually caused by either alkaline or acidic agents (Cortina, et al. Applications, Keratoprostheses and Artificial Corneas. 2015^{th} Ed.). Although both are serious injuries, alkaline burns are more common (Wagoner, et al. Survey of ophthalmology 1997 41:275-313) and cause more severe damage than acid burns (Rozenbaum, et al. Burns. 1991 17(2):136-40). The systemic response to tissue damage and the subsequent wound healing response typically results in induction of inflammatory cascades. Neutrophils provide the first response to tissue damage and may have dual functions. Initially, the infiltration of neutrophils into injured tissue protects wounds from invading pathogens and clears debris (Theilgaard-Mönch, et al. J Immunol. 2004 172(12):7684-93). However, due to short half-life of infiltrating neutrophils, dead neutrophils can release their nuclear and granular contents, known as neutrophil extracellular traps (NETs). Release of NETs can inhibit keratinocyte migration, possibly proliferation (Dovi, et al. J Leukoc Biol. 2003 73(4):448-55), and impair the wound healing process (Huang, Jet al. Cell Death Discov. 2020 6:84; Wan, et al. Front Immunol. 2020 11:551057). Thus, understanding the molecular mechanisms of neutrophil death is critical for the development of potential treatments that enhance tissue repair and suppress the potential detrimental actions of neutrophils following corneal injury.

### SUMMARY

Disclosed herein are methods for treating ocular chemical burns in a subject that involves administering to the cornea of a subject in need thereof a composition comprising a pyroptosis inhibitor, such as disulfiram.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1H show GsdmD−/− corneas heal faster and develop less neovascularization after alkali induced injury. FIG. 1A contains representative images of fluorescein uptake showed that re-epithelialization was improved in the corneas of GsdmD−/− mice as compared to WT mice (n=5 for GsdmD−/− mice, n=7 for WT mice). FIG. 1B shows quantification of fluorescein signal. FIG. 1C contains representative images of H&E and immunofluorescence staining showed that GsdmD−/− corneas displaced improved re-epithelialization with significantly more epithelial cell layers one day after injury. MPO staining indicated that more neutrophil infiltrated in the GsdmD−/− mice as compared to WT corneas. FIG. 1E shows quantification of epithelial cell layers. FIG. 1E shows at day 10 post alkali injury, GsdmD−/− corneas developed significantly less corneal neovascularization as compared to WT corneas. FIG. 1F shows neovascularization scores at indicated time points. FIG. 1G shows CD31 staining of flat mount corneas showed that corneas derived from GsdmD−/− mice had less vascularization than those in WT mice. FIG. 1H shows quantification of CD31 fluorescent intensity in panel G.

FIG. 2A to 2C show neutrophils infiltrate into injured corneas and undergo pyroptosis. FIG. 2A shows Western blotting analysis showed expression of MPO, GsdmD protein in the injured corneas of WT mice at indicated timepoints after alkali injury. FIG. 2B contains representative immunofluorescent images of flat mount mouse corneas were staining with cleaved GsdmD (GsdmD-N), F4/80 and MPO. Co-localization of MPO and GsdmD-N was detected. FIG. 1C shows WT mice receiving Gr-1 antibody treatments (two i.p. injections, 100 μg/mouse at 16 and 0 hours before alkaline injury) showed significantly reduction of MPO and GsdmD-N expression at 24 hours after alkali induced injury.

FIGS. 3A to 3E show pyroptotic neutrophils inhibit corneal epithelial cell migration and disrupt their integrity. FIG. 3A shows corneas from indicated treatments were incubated with corneal epithelial cells. Scratch wounds were induced and representative images were taken at 0 and 6 hrs after scratch wound. FIG. 3B shows quantification of scratch wounds in FIG. 3A. FIG. 3C shows co-cultured of isolated neutrophil derived from WT mice but not Gsdmd−/− mice inhibited migration of corneal epithelial cells. FIG. 3D shows quantification of migration rates. FIG. 3E shows co-culture of WT neutrophils with corneal epithelial cells disrupted epithelial integrity as measured by TEER.

FIG. 4. IL-1β and IL-18 released from pyroptotic neutrophils inhibit corneal epithelial cell migration and disrupt their integrity. (A) Treatment of recombinant activated IL-1β and IL-18 proteins could significantly suppress corneal epithelial cell migration (pro and boiled recombinant IL-1β and IL-18 proteins were used as controls) (B) Migration rates of A were quantified. (C) TEER measurements showed that activated IL-1β and IL-18 could also significantly reduce the electrical resistance of corneal epithelial cells. (D) ZO-1 staining showed that the tight junction was disrupted after treatments of activated IL-1β and IL-18 recombinant proteins (fluorescent signal of ZO-1 in different groups were quantified in E).

FIGS. 5A to 5C show Wnt5A expresses in limbus of uninjured corneas and newly regenerated corneal epithelium. FIG. 5A shows mRNA expressions of several known macrophage markers in corneas derived from WT and GsdmD−/− mice at 24 hours after injury. FIG. 5B shows immunofluorescent staining on cross-section slides of mouse eyes showed Wnt5A expressed highly in the newly differentiated corneal epithelial cells after injury. FIG. 5C shows flat mount staining of mouse corneas showing that Wnt5A mainly expressed in limbus area before injury. After alkali injury, Wnt5A expressed in newly generated epithelial cells. Macrophages (F4/80) and neutrophils (MPO) were also observed after injury.

FIGS. 6A and 6B show sFlt-1 highly expresses in newly generated corneal epithelium after injury. FIG. 6A shows mRNA expression of VEGFa, VEGFR1, VEGFR2 and sflt-1 were quantified by real-time RT-PCR in corneas of WT and GsdmD−/− mice at 24 hours after injury (n=5 for each group). FIG. 6B shows sFlt-1 staining showed that sFlt-1 was expressed in corneal epithelial cells before injury and expression of sFlt-1 in epithelial cells was enhanced after corneal injury.

FIGS. 7A to 7H show inhibition of pyroptosis enhances corneal wound healing and inhibits post-injury corneal neovascularization. FIG. 7A shows bone marrow cells from WT and GsdmD−/− mice were transplanted to the irradiation treated WT mice. Mice received GsdmD−/− bone marrows showed improved re-epithelialization (quantified in FIG. 7B). FIG. 7C shows dual antibodies treatment to deplete neutrophils as well as disulfiram treatment to inhibit pyroptosis (FIG. 7E) could also promote the re-epithelialization (quantified in FIGS. 7D and 7F). FIG. 7G shows CD31 staining of corneal flat mount showed that disulfiram treatment could significantly reduce neovascularization after corneal injury (quantified in FIG. 7H).

FIG. 8 contains a schematic figure summarizes the role of neutrophil pyroptosis in regulating corneal wound healing and post-injury neovascularization.

FIG. 9 shows clinical opacity scores showing no difference between GsdmD−/− and WT corneas after injury.

FIG. 10 shows combination of LPS and Nigericin fail to induce pyroptosis in mouse primary cultured epithelial cells (mPCEC).

FIG. 11 shows primary cultured neutrophils first undergo pyroptosis and release IL-1β, followed by apoptosis (cleaved caspase 3).

FIG. 12 shows treatment of disulfiram inhibits cleavage of GsdmD but not cleavage of Caspase-3 in primary cultured neutrophil.

FIG. 13 shows IF staining showed increased IL-1β signal after injury and this signal mainly co-localized with MPO in the cornea 1 day after injury.

FIG. 14 shows realtime RT-PCR showed upregulation of VEGFa in both WT and GsdmD−/− corneas in similar level.

FIG. 15 shows the expression of GsdmD in the spleen and bone marrow cells flushed from WT mice that received bone marrow transplantation from WT and GsdmD−/− mice, respectively. Ponceau S were used as internal control.

FIG. 16 shows Western blotting analysis confirms depletion of neutrophils after antibodies injection at indicated time points.

FIG. 17 shows treatment of disulfiram could suppress cleavage of both GsdmD and Caspase-1.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “prevent” refers to a treatment that forestalls or slows the onset of a disease or condition or reduced the severity of the disease or condition. Thus, if a treatment can treat a disease in a subject having symptoms of the disease, it can also prevent that disease in a subject who has yet to suffer some or all of the symptoms.

### Corneal Chemical Burns

Chemical burns are the result of exposure to a variety of substances commonly found at home, the workplace, and the surrounding environment. The burn may be obvious, for example, from a direct spill or other exposure, or more covert, especially in children. Chemical burns can cause short-term, long-term, and lifelong health problems, especially if undertreated. Occasionally, they can result in premature death, especially if ingested in an attempt to self-harm.

Common causes of chemical burns include acids, bases (alkali agents), oxidants, and vesicants (blister agents). Acids includes sulfuric, nitric, hydrofluoric, hydrochloric, acetic acid, formic, phosphoric, phenols, and chloroacetic acid.

Alkali bases include sodium and potassium hydroxide, calcium hydroxide, sodium and calcium hypochlorite, ammonia, phosphates, silicated, sodium carbonate, and lithium hydride. This includes, for example, lime, lye, ammonia, and drain cleaners. Oxidants include bleaches like chlorites used in the home, peroxides, chromates, and magnates.

Vesicants (blister agents)include, but are not limited to, sulfur mustard, nitrogen mustard, and phosgene oxime. Toxic or injurious effects of blister agents include pain, irritation, and/or tearing in the skin, eye, and/or mucous, and conjunctivitis and/or corneal damage to the eye. Sulfur mustard is the compound bis(2-chlorethyl) sulfide. Nitrogen mustard includes the compounds bis(2-chlorethyl)ethylamine, bis(2-chlorethyl)methylamine, and tris(2-chlorethyl)amine.

### Pyroptosis Inhibitors

The pore-forming protein gasdermin (such as gasdermin D) is the final pyroptosis executioner downstream of inflammasome activation. pyroptosis inhibitor compounds of the present disclosure inhibit gasdermin D pore formation and subsequent secretion of inflammatory mediators such as IL-1β and IL-18.

In embodiments, the pyroptosis inhibitor is Disulfiram (also known as Tetraethylthiuram disulfide; CAS Reference Number: 97-77-8).

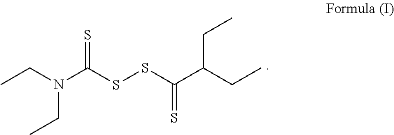 [SMILES: CCC(CC)C(=S)SSC(=S)N(CC)CC]

Disulfiram is a specific inhibitor of an aldehy de-dehydrogenase (ALDH1). Disulfiram was approved by the US Food and Drug Administration in 1951 for alcohol aversion therapy after researchers observed that it induced the effects of a hangover after alcohol consumption.

As used herein, the term disulfiram includes disulfiram itself and any pharmaceutically acceptable salt, solvate, or prodrug thereof. As used herein, the term disulfiram includes disulfiram itself and any of its metabolites and/or their derivatives and variants thereof. Examples of metabolites include diethyldithiocarbamate, methyl diethyldithiocarbamate, dithiomethylcarbamate, S-Methyl N,N-Diethylthiocarbamate, diethyl-amine, and carbon disulfide.

As used herein, the term disulfiram includes disulfiram itself and any of its metabolites and/or their derivatives and variants thereof. The term disulfiram includes variants of disulfiram, which, as used herein is, a compound having the structure of Formula (II), or a pharmaceutically acceptable salt, solvate, or prodrug thereof:

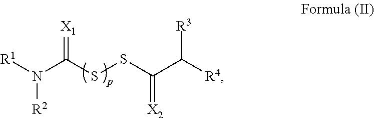

wherein,

X1 and X2 are each independently O or S;

p is 1 or 2;

R¹, R², R³, and R⁴ are each independently H, C₁-C₆ alkyl, C₁-C₆ haloalkyl, C₂-C₆ alkenyl, C₂-C₆ alkynyl, C₃-C₈ cycloalkyl, 4-10 membered heterocycloalkyl, C(O)OR^{1a}, or C(O)NR^{1a}R^{1b}; wherein each is optionally substituted with 1, 2, or 3 R⁵;

or R¹ and R² together with the N atom to which they are attached from a 4-10 membered ring, which is optionally substituted with 1, 2, 3, or 4 R⁶;

or R³ and R⁴ together with the N atom to which they are attached from a 4-10 membered ring, which is optionally substituted with 1, 2, 3, or 4 R⁷; each R⁵, R⁶, and R⁷ is independently selected from halogen, C₁-C₆ alkyl, C₁-C₆ haloalkyl, CN, NO₂, OR^{1a}, C(O)R^{1b}, C(O)NR^{1a}R^{1b}, C(O)OR^{1a}, NR^{1a}R^{1b}, S(O)2R^{1b} and S(O)₂NR^{1a}R^{1b}; each R^{1a} and R^{1b} is independently selected from H, C₁-C₆ alkyl, C₁-C₆ alkenyl, C₁-C₆ alkynyl, cycloalkyl, heterocycloalkyl, aryl or heteroaryl.

In some embodiments, the pyroptosis inhibitor is combined (in a composition and/or in a method) with a potentiating ingredient (e.g., tert-Butylhydroquinone (TBHQ), cinnamaldehyde, or nordihydroguaiaretic acid (NDGA). In some embodiments, the potentiating ingredient, as used in the present disclosure enhances, increases, and/or improves the effectiveness and/or desirable activity of the active ingredient.

In some embodiments, the pyroptosis inhibitor is combined with a steroid. Steroids for ocular administration may include, but are not limited to, triamcinolone (Aristocort®; Kenalog®), betamethasone (Celestone®), budesonide, cortisone, dexamethasone (Decadron-LA®; Decadron® phosphate; Maxidex® and Tobradex® (Alcon)), hydrocortisone, methylprednisolone (Depo-Medrol®), Solu-Medrol®), prednisolone (prednisolone acetate, e.g., Pred Forte® (Allergan); Econopred and Econopred Plus® (Alcon); AK-Tate® (Akorn); Pred Mild® (Allergan); prednisone sodium phosphate (Inflamase Mild and Inflamase Forte® (Ciba); Metreton® (Schering); AK-Pred® (Akorn)), fluorometholone (fluorometholone acetate (Flarex® (Alcon); Eflone®), fluorometholone alcohol (FML® and FML-Mild®, (Allergan); FluorOP®)), rimexolone (Vexol®((Alcon)), medrysone alcohol (HMSO (Allergan)); Iotoprednol etabonate (Lotemax® and Alre)(® (Bausch & Lomb), 11-desoxcortisol, and anecortave acetate (Alcon)). It will be appreciated that the above lists are representative only and are not exclusive.

### Formulations

Compositions of the present disclosure are formulated to be suitable for in vivo administration to a mammal. Such compositions can optionally comprise a suitable amount of a pharmaceutically acceptable excipient so as to provide the form for proper administration. Pharmaceutical excipients can be aqueous liquids, such as water or saline. Pharmaceutical excipients can be lipid based, e.g., comprising a liquid or solid oil. In addition, auxiliary, stabilizing, thickening, lubricating, and coloring agents can be used. The pharmaceutically acceptable excipients are sterile when administered to a subject. Water is a useful excipient when any composition described herein is administered parentally or in some oral formulations. In embodiments, the compositions described herein are suspended in a saline buffer (including, without limitation Ringer's, TBS, PBS, HEPES, HBSS, and the like). Saline solutions and aqueous dextrose and glycerol solutions can also be employed as liquid excipients, specifically for injectable solutions. Suitable pharmaceutical excipients also include starch, glucose, lactose, sucrose, glycerol monostearate, mannitol, sodium chloride, dried skim milk, glycerol, propylene, glycol, water, ethanol and the like. Any composition described herein, if desired, can also comprise pH buffering agents.

In addition, the pharmaceutical formulation may include additives such as, for example, other buffers, diluents, carriers, adjuvants or excipients. Any pharmacologically acceptable buffer suitable for application to the eye may be used, e.g., tris or phosphate buffers. Other agents may be employed in the formulation for a variety of purposes. For example, buffering agents, preservatives, co-solvents, surfactants, oils, humectants, emollients, chelating agents, stabilizers or antioxidants may be employed. Water soluble preservatives which may be employed may include, but are not limited to, benzalkonium chloride, chlorobutanol, thimerosal, sodium bisulfate, phenylmercuric acetate, phenylmercuric nitrate, ethyl alcohol, methylparaben, polyvinyl alcohol, benzyl alcohol and phenylethyl alcohol. A surfactant may be Tween 80. Other vehicles that may be used include, but are not limited to, polyvinyl alcohol, povidone, hydroxypropyl methyl cellulose, poloxamers, carboxymethyl cellulose, hydroxyethyl cellulose, purified water, etc. Tonicity adjustors may be included, for example, sodium chloride, potassium chloride, mannitol, glycerin, etc. Antioxidants may include, but are not limited to, sodium metabisulfite, sodium thiosulfate, acetylcysteine, butylated hydroxyanisole, butylated hydroxytoluene, etc. The indications, effective doses, formulations, contraindicatons, vendors etc., of the compounds in the formulations are available or are known to one skilled in the art.

These agents may be present in individual amounts of from about 0.001 to about 5% by weight and preferably about 0.01% to about 2%. Suitable water soluble buffering agents that may be employed are sodium carbonate, sodium borate, sodium phosphate, sodium acetate, sodium bicarbonate, etc., as approved by the US FDA for the desired route of administration. These agents may be present in amounts sufficient to maintain a pH of the system of between about 2 to about 9 and preferably about 4 to about 8. As such the buffering agent may be as much as about 5% on a weight to weight basis of the total formulation. Electrolytes such as, but not limited to, sodium chloride and potassium chloride may also be included in the formulation.

The dosage of any herein—disclosed composition or compositions can depend on several factors including the characteristics of the mammal to be administered. Examples of characteristics include species, strain, breed, sex, age, weight, size, health, and/or disease status. Moreover, the dosage may depend on whether the administration is the first time the subject received a composition of the present disclosure or if the subject has previously received a composition of the present disclosure. Additionally, pharmacogenomic (the effect of genotype on the pharmacokinetic, pharmacodynamic or efficacy profile of a composition) information about a particular subject may affect dosage used. Furthermore, the exact individual dosages can be adjusted somewhat depending on a variety of factors, including the specific composition being administered, the time of administration, the route of administration, the nature of the formulation, and the rate of excretion. Some variations in the dosage can be expected.

Moreover, the dosage may depend on the specific ingredients administered. Disulfiram may be encapsulated in one microcapsule and the one or more additional ingredients may be encapsulated into another microcapsule. Disulfiram and the one or more additional ingredients may be encapsulated into one microcapsule. Disulfiram may be encapsulated in one microcapsule and the one or more additional ingredients may not be encapsulated. Disulfiram may not be encapsulated and the one or more additional ingredients are encapsulated in a microcapsule. The microcapsule may be a liposome, an albumin microsphere, a microemulsion, a nanoparticle (e.g., a lipid nanoparticle), and a nanocapsule. In embodiments, microcapsules, e.g., lipid nanoparticles and liposome∧ include lipids selected from one or more of the following categories: cationic lipids; anionic lipids; neutral lipids; multi-valent charged lipids; and zwitterionic lipids. In some cases, a cationic lipid and/or cationic polymer may be used to facilitate a charge-charge interaction with the active ingredient (disulfiram) and the potentiating ingredient (TBHQ). The microcapsule may comprise a PEGylated lipid. Examples of microcapsules and methods for manufacturing the same are described in the art. See, e.g., Prui et al. , Crit Rev Ther Drug Carrier Syst., 2009; 26(6): 523-580; Wakasar, J Drug Target, 2018, 26(4):311 -318, Langer, 1990, Science 249: 1527-1533; Treated al. , in “Liposomes in the Therapy of Infectious Disease and Cancer”, Lopez-Berestein and Fidler (eds.), Liss, New York, pp. 353-365 (1989); Pelaz et al. “Diverse applications of nanomedicine.” (2017): 2313-2381; the contents of each of which is incorporated herein by reference in its entirety.

### Administration

Any of the formulations may be administered by an ocular route, such as topical, subconjunctival, sub-Tenon, intraocular, etc. Moreover the formulation may be administered as a slow release formulation, with a carrier formulation such as microspheres, microcapsules, liposomes, etc., as an intravenous solution or suspension, or in an intraocular injection, as known to one skilled in the art. A time-release drug delivery system may be administered intraocularly to result in sustained release of the agent over a period of time. The formulation may be in the form of a vehicle, such as a micro- or macro-capsule or matrix of biocompatible polymers such as polycaprolactone, polyglycolic acid, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAIB), and other polymers such as those disclosed in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety, or lipids that may be formulated as microspheres or liposomes. A microscopic or macroscopic formulation may be administered through a needle, or may be implanted by suturing within the eye, for example, within the lens capsule. Delayed or extended release properties may be provided through various formulations of the vehicle (coated or uncoated microsphere, coated or uncoated capsule, lipid or polymer components, unilamellar or multilamellar structure, and combinations of the above, etc.). The formulation and loading of microspheres, microcapsules, liposomes, etc. and their ocular implantation are standard techniques known by one skilled in the art, for example, the use a ganciclovir sustained-release implant to treat cytomegalovirus retinitis, disclosed in Vitreoretinal Surgical Techniques, Peyman et al., Eds. (Martin Dunitz, London 2001, chapter 45); Handbook of Pharmaceutical Controlled Release Technology, Wise, Ed. (Marcel Dekker, N.Y. 2000), the relevant sections of which are incorporated by reference herein in their entirety. For example, a sustained release intraocular implant may be inserted through the pars plana for implantation in the vitreous cavity. An intraocular injection may be into the vitreous (intravitreal), or under the conjunctiva (subconjunctival), or behind the eye (retrobulbar), or under the Capsule of Tenon (sub-Tenon), and may be in a depot form. Other intraocular routes of administration and injection sites and forms are also contemplated and are within the scope of the invention.

Administration of the inventive formulation should at least reduce ocular neovascularization. Vessel regression may occur in addition to, or in place of, prevention of further vessel growth or proliferation. As will be appreciated, the cumulative effects may be important in managing diseases such as diabetes, where control of the complicating factors of the disease is as important as control of the underlying pathology to maintain a patient's quality of life.

Topical application of formulations may be as an in situ gellable aqueous formulation. Such a formulation comprises a gelling agent in a concentration effective to promote gelling upon contact with the eye or with lacrimal fluid in the exterior of the eye. Suitable gelling agents may include, but are not limited to, thermosetting polymers such as tetra-substituted ethylene diamine block copolymers of ethylene oxide and propylene oxide (e.g., poloxamine); polycarbophil; and polysaccharides such as gellan, carrageenan (e.g., kappa-carrageenan and iota-carrageenan), chitosan and alginate gums.

The phrase “in situ gellable” as used herein embraces not only liquids of low viscosity that form gels upon contact with the eye or with lacrimal fluid in the exterior of the eye, but also more viscous liquids such as semi-fluid and thixotropic gels that exhibit substantially increased viscosity or gel stiffness upon administration to the eye. Indeed, it can be advantageous to formulate a formulation of the invention as a gel, to minimize loss of the formulation immediately upon administration, as a result, for example, of lacrimation caused by reflex blinking. Although it is preferred that such a formulation exhibit further increase in viscosity or gel stiffness upon administration, this is not absolutely required if the initial gel is sufficiently resistant to dissipation by lacrimal drainage to provide the effective residence time specified herein.

To prepare a topical formulation for the treatment of ophthalmological disorders, a therapeutically effective amount of the formulation of the invention is placed in an ophthalmological vehicle as is known in the art. The amount of the therapeutic compound to be administered and the concentration of the compound in the topical formulations depend upon the diluent, delivery system or device selected, the clinical condition of the patient, the side effects and the stability of the compound in the formulation. Thus, the physician employs the appropriate preparation containing the appropriate concentration of the therapeutic compound and selects the amount of formulation administered, depending upon clinical experience with the patient in question or with similar patients.

Where the formulation contains two or more active agents, the active agents may be administered as a mixture, as an admixture, in the same formulation, in separate formulations, in extended release formulations, liposomes, microcapsules, or any of the previously described embodiments.

The formulation may be administered topically, or may be injected into the eye, or one active agent may be administered topically and the other agent(s) may be injected.

In certain embodiments, disulfiram is administered at a daily dosage or as a single dosage of about 5 mg to about 750 mg per day. For example, disulfiram is administered at a total daily dosage or as a single dosage of about 5, 6, 7, 8, 9, 10, 15, 18, 18.5, 19, 20, 25, 30, 45, 40, 45, 50, 55, 60, 65, 70, 75, 80, 85, 90, 95, 100, 125, 150, 175, 200, 225, 240, 250, 260, 275, 300, 325, 350, 375, 400, 425, 450, 475, 500 mg, 525 mg, 550 mg, 575 mg, 600 mg, 625 mg, 650 mg, 675 mg, or 700 mg per day, and any total daily dosage or single dosage therebetween. As examples, the total daily dosage or the single dosage may be 5-10 mg, 10-15 mg, 15-20 mg, 20-25 mg, 25-30 mg, 30-35 mg, 35-40 mg, 40-45 mg, 45-50 mg, 50-55 mg, 55-60 mg, 60-65 mg, 65-70 mg, 70-75 mg, 75-80 mg, 80-85 mg, 85-90 mg, 90-95 mg, 95-100 mg, 100-125 mg, 125-150 mg, 150-175 mg, 175-200 mg, 200-225 mg, 225-250 mg, 230-260 mg, 240-270 mg, 250-275 mg, 275-300 mg, 300-325 mg, 325-350 mg, 350-375 mg, 375-400 mg, 400-425 mg, 425-450 mg, 450-475 mg, 475-500 mg, 500-525 mg, 525-550 mg, 550-575 mg, 575-600 mg, 600-625 mg, 625-650 mg, 630-660 mg, 640-670 mg, 650-675 mg, or 675-700 mg, and any total daily dosage or single dosage therebetween. The total daily dosage or the single dosage may be 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, or 300 mg, and any total daily dosage or single dosage therebetween. In some embodiments, the total daily dosage or the single dosage is between 230 mg and 260 mg, e.g., 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, or 260 mg, and any total daily dosage or single dosage therebetween. Is some embodiments, the total daily dosage or the single dosage is 250 mg. The total daily dosage or the single dosage may be 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, or 500 mg, and any total daily dosage or single dosage therebetween Is some embodiments, the total daily dosage or the single dosage is 500 mg. The total daily dosage or the single dosage may be 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, or 750 mg, and any total daily dosage or single dosage therebetween Is some embodiments, the total daily dosage or the single dosage is 750 mg. In some embodiments, the total daily dosage or the single dosage of disulfiram is 250 mg. The disulfiram may be administered 1×, 2×, or 3× per day to achieve the daily dosage. Thus, for a daily dose of 250 mg with a once per day administration, only a single administration of 250 mg will be given; for a daily dose of 500 mg with a twice per day administration, two administrations of about 250 mg will be given; and for a daily dose of 750 mg with a thrice per day administration, three administrations of about 250 mg will be given. Similarly, for a daily dose of 500 mg with a once per day administration, only a single administration of 500 mg will be given; for a daily dose of 500 mg with a twice per day administration, two administrations of about 250 mg will be given; and for a daily dose of 500 mg with a thrice per day administration, three administrations of about 170 mg will be given.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Neutrophil Pyroptosis Regulates Corneal Wound Healing and Post-injury Neovascularization

### Introduction

Neutrophils have been shown to be the main source of IL-1β production following injury (Mankan, et al. Eur J Immunol. 2012 42(3):710-5; Ryu, et al. Mucosal Immunol. 2017 10(3):757-774). Caspase-1 plays a major role in the activation of the proinflammatory cytokines IL-1β and IL-18 in the neutrophil (Fantuzzi, et al. Blood. 1998 91(6):2118-25). Caspase-1/11 knockout and IL-1β and IL-18 knockout can improve the survival rate in both the cecal ligation and puncture and septic shock mouse models (Sarkar, et al. Am J Respir Crit Care Med. 2006 174(9):1003-10; Wang, et al. Int J Med Microbiol. 2017 307(8):490-496; Vanden Berghe, et al. Am J Respir Crit Care Med. 2014 189(3):282-91). These studies highlight the important role of neutrophil cell death induced inflammatory cytokine release in disease states.

Pyroptosis, a lytic form of cell death, is a key pathway triggering inflammation (Cookson. Trends Microbiol 2001 9:113-114; Liu, et al. Nat Rev Drug Discov 2021 20:384-405; Man, et al. Immunol Rev 2017 277:61-75; Bergsbaken, et al. Nat Rev Microbiol 2009 7:99-109). Gasdermin D (GsdmD) has been confirmed as the key effector leading to pyroptosis and NETosis (Shi, et al. Nature 2015 526:660-665; Kayagaki, et al. Nature 2015 526:666-671). Neutrophil NETosis requires GsdmD formed pores for the rupture of the plasma membrane and granule, with subsequent NET extrusion (Liu, et al. Nat Rev Drug Discov 2021 20:384-405; Sollberger, et al. Science immunology 2018 3). In addition to activation of IL-1β and IL-18, caspase-1 is required and involved in the cleavage of GsdmD (Shi, et al. Nature 2015 526:660-665; Karmakar, et al. Nature communications 2020 11:2212; Liu, et al. Cell Mol Life Sci. 2019 76(11):2031-2042). Recently, cleavage of GsdmD and induction of neutrophil pyroptosis via caspase-11 and neutrophil elastase has also been reported (Karmakar, et al. J Immunol. 2015 194(4):1763-75; Chen, et al. Sci Immunol. 2018 3(26):eaar6676; Kambara, et al. Cell Rep. 2018 22(11):2924-2936). However, the involvement of neutrophil pyroptosis in corneal wound healing is largely unknown.

This Example shows that genetic ablation of GsdmD (GsdmD^{-/-}) could promote corneal wound healing and reduce subsequent corneal neovascularization (CNV). Mechanistically, IL-1β and IL-18 released from pyroptotic neutrophils suppressed migration of corneal epithelial cell and delayed corneal healing. Furthermore, two key molecules were identified that inhibit corneal angiogenesis, Wnt5a and sflt-1, both of which were highly expressed in the newly differentiated epithelial cell after injury. Thus, inhibiting neutrophil pyroptosis and promoting sflt-1 facilitates timely corneal reepithelization and suppression of post-injury neovascularization. These study highlights the importance of neutrophil pyroptosis in corneal wound healing and neovascularization; pyroptosis may be a potential target for developing effective means to treat corneal wounds.

### Results

### GsdmD Deficiency Promotes Corneal Wound Healing and Reduces Neovascularization.

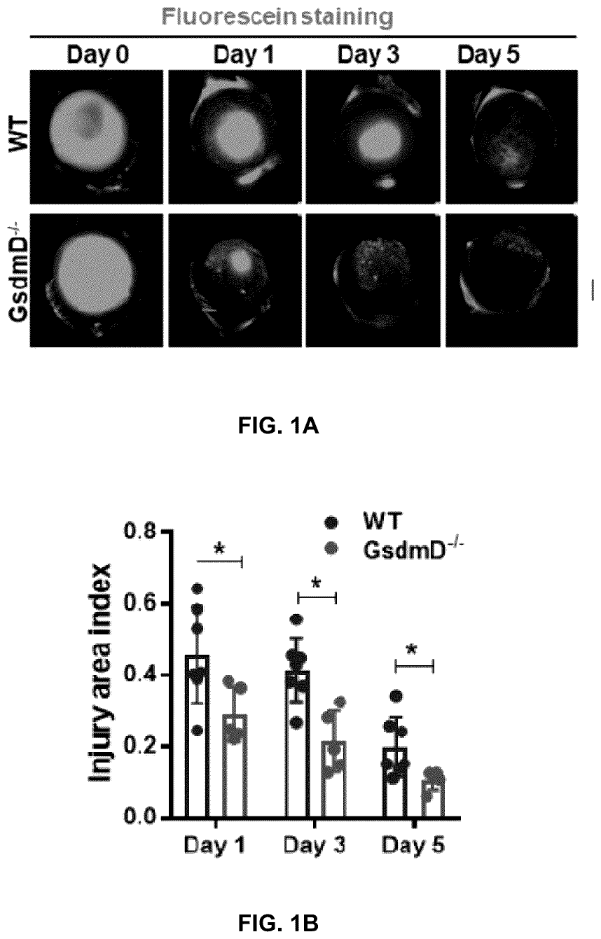

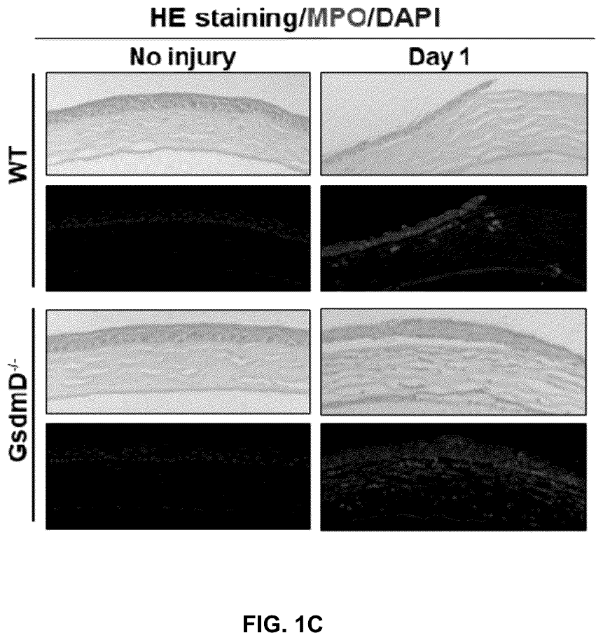

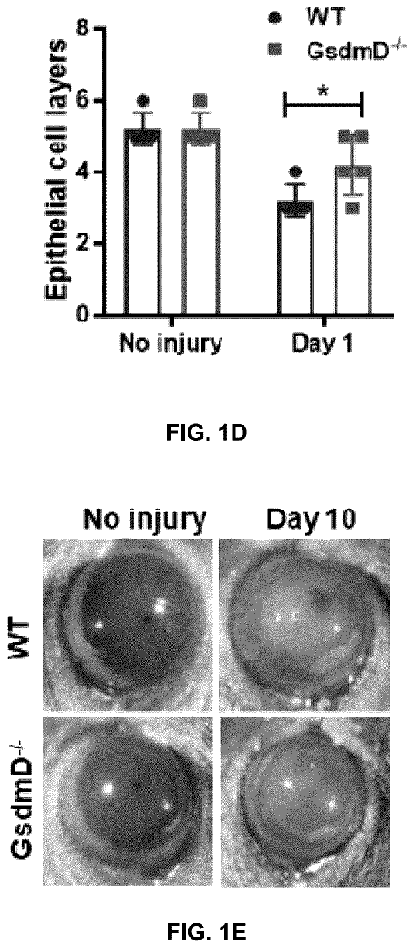

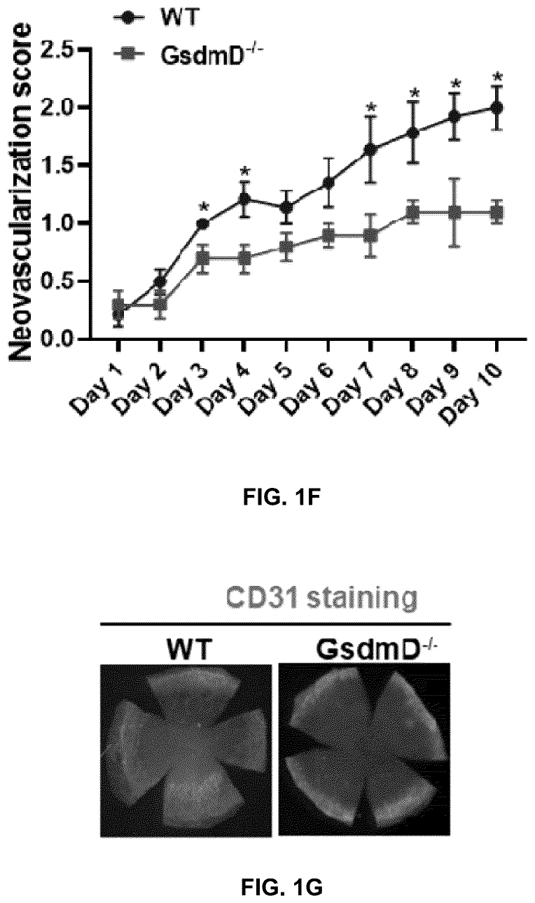

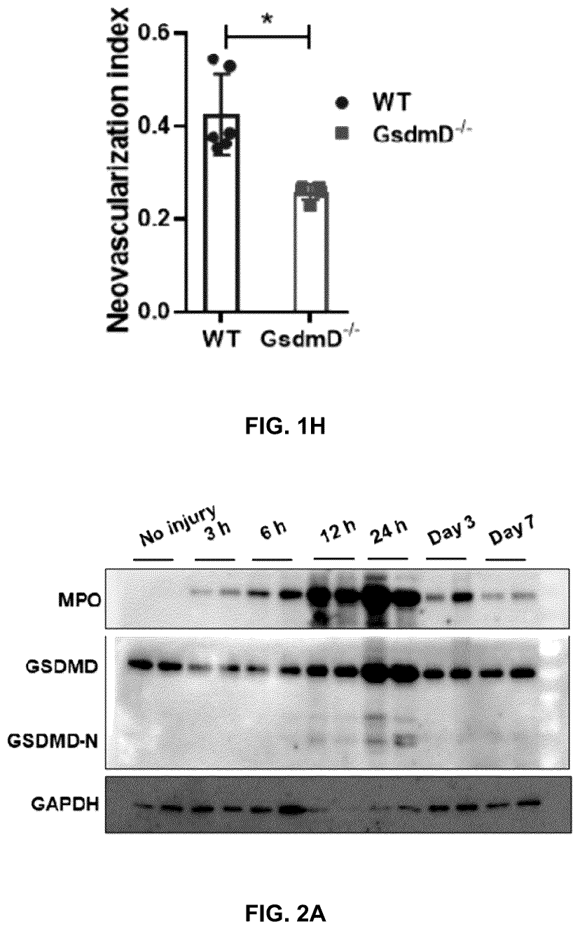

To study the potential role of pyroptosis in corneal wound healing, GsdmD deficient (GsdmD−/−) mice were subjected to alkali induced corneal injury (Chandler, et al. Commun Biol. 2019 2:71). Twenty-four hours after injury, corneal fluorescein staining demonstrated that GsdmD^{-/-} mice (28.89%±7.63%) had significantly smaller wound areas than that of WT mice (45.57%±13.60%) (FIGS. 1A and 1B). Consistent with the fluorescein staining, H&E and DAPI immunofluorescent (IF) staining also showed improved corneal re-epithelization in the GsdmD^{-/-} mice (FIGS. 1C and 1D). The number of epithelial cell layers in the GsdmD KO mice (4.2±0.8) was significantly higher than that of WT mice (3.2±0.4).

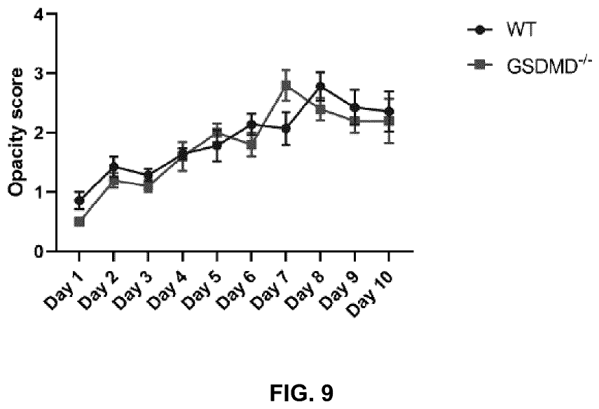

Alkali injury is a well-established model for the induction of CNV(Anderson, et al. J Vis Exp. 2014 (86):51159). After injury, limbal blood vessels are stimulated to grow centrally towards the axial cornea, compromising vision. Following injury induction, healing and vascular encroachment were examined daily until day 10. Slit lamp pictures of mouse corneas clearly show that CNV was significantly reduced in GsdmD−/− corneas compared to WT corneas (FIG. 1E and FIG. 1F). CD31 staining of flat mount corneas further confirmed a reduction of CNV in injured GsdmD^{-/-} corneas (FIG. 1G and FIG. 1H). Interestingly, there were no significant differences in fibrosis between WT and GsdmD^{-/-} mice (FIG. 9). Thus, these findings suggest that pyroptosis plays a detrimental role in corneal re-epithelialization and vascularization but may not be involved in mitigating stromal fibrosis.

Neutrophils are the Main Cell Type that Undergo Pyroptosis After Corneal Wounding.

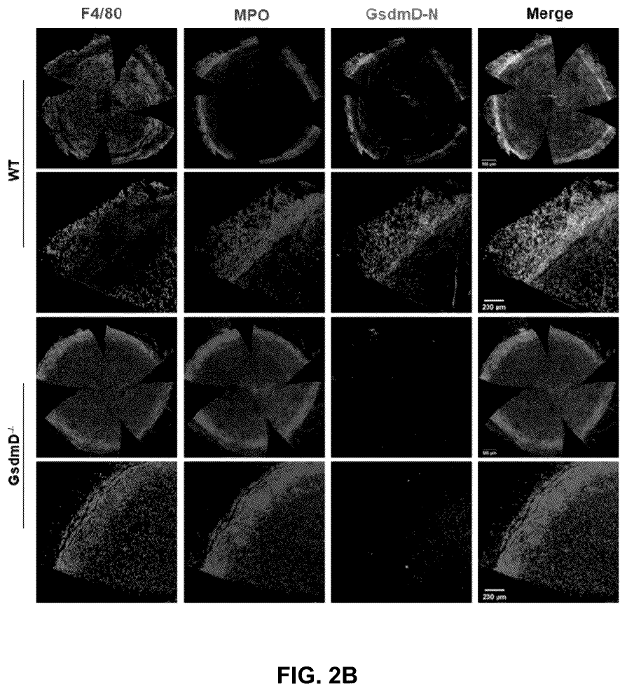

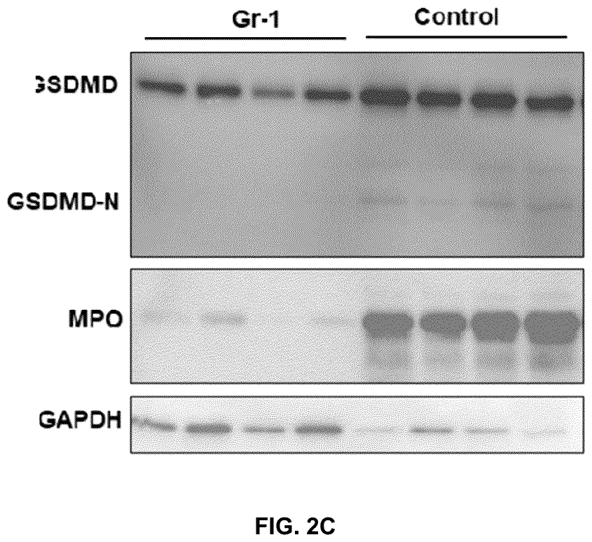

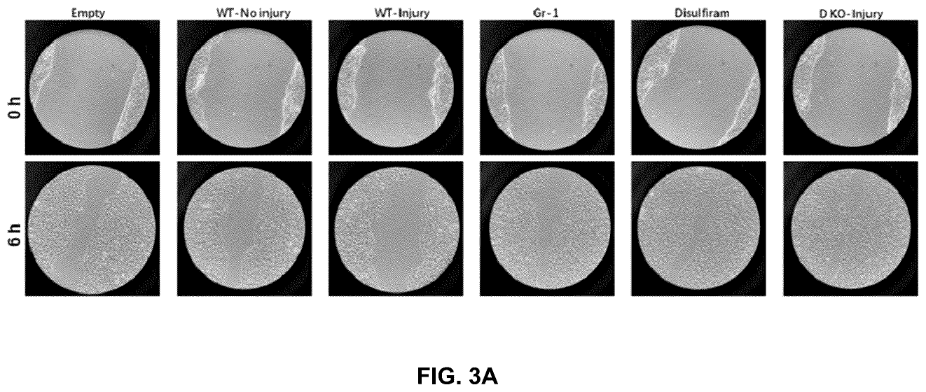

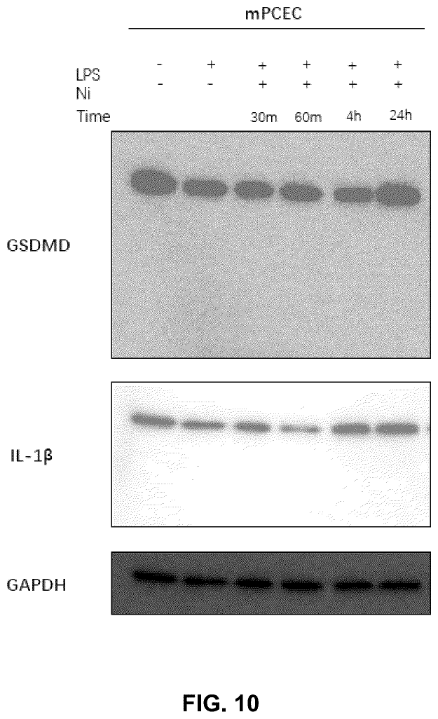

As the corneal epithelium is the initial cell type affected by alkali injury, the first thing tested was whether corneal epithelial cells underwent pyroptosis. However, pyroptosis was not induced in primary cultured mouse corneal epithelial cells (mCEC) with a combination of lipopolysaccharide (LPS) and nigericin (Ni) treatment (FIG. 10). To further delineate the source of pyroptosis after corneal wounding, the dynamics of pyroptosis after corneal injury was next determined. As shown in FIG. 2A, activation of pyroptosis (cleaved GsdmD: GsdmD-N) was observed as early as 12 hrs after injury and expression peaked at 24 hrs. Interestingly, when the same samples were probed with myeloperoxidase (MPO, a neutrophil marker), neutrophil infiltration was observed as early as 3 hrs after injury and peaked at the same time (24 hrs) as pyroptosis (FIG. 2A), indicating a possible link between neutrophil infiltration and pyroptosis. Costaining for F4/80 (a macrophage marker), MPO, and GsdmD-N demonstrated that activation of pyroptosis co-localized with neutrophils, but not macrophages (FIG. 2B). Additionally, neutrophils were localized mainly around the limbus in WT mice at 24 hrs after injury, while a greater number and wider distribution (both limbus and cornea) of neutrophils was detected in GsdmD−/− corneas (FIG. 2B). To determine the extent that neutrophils contributed to post-injury pyroptosis in the cornea, we then injected the mice with Gr-1 antibody to deplete neutrophils prior to injury (Suzuki, et al. Sci Rep. 2022 12(1):4136). As shown in FIG. 2C, depletion of neutrophils almost completely abolished activation of pyroptosis, as evidenced by lack of GsdmD-N signal. Taken together, these results indicate that alkali injury-induced pyroptosis largely occurred in infiltrating neutrophils and inhibition of pyroptosis led to greater neutrophil survival in the injured cornea.

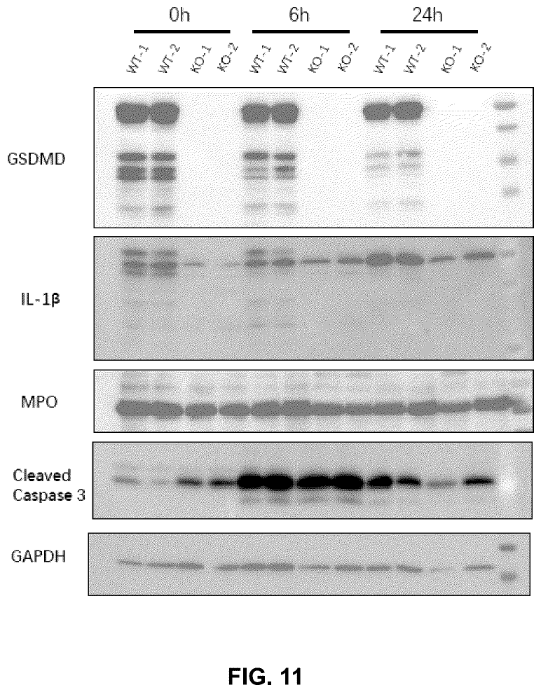

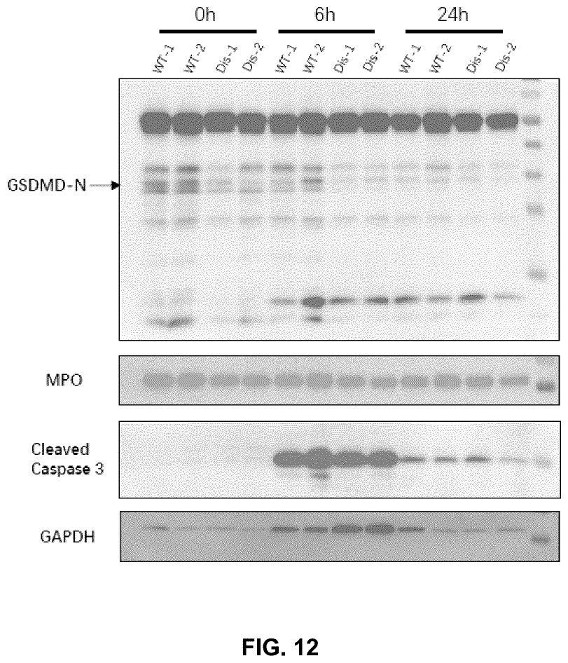

To further confirm neutrophils undergo pyrotposis, primary neutrophils were isolated and purified from mouse bone marrow. Isolated neutrophils underwent pyroptosis quickly after isolation followed by activation of apoptosis, as evidenced by cleaved caspase-3 (FIG. 11). Treatment with disulfiram (a reported pyroptosis inhibitor) could significantly inhibit the GsdmD cleavage, but not Caspase-3 cleavage, in isolated neutrophils (FIG. 12). Thus, these findings demonstrate that pyroptosis is a major type of cell death in neutrophils.

### Neutrophil Pyroptosis Delays Epithelial Cell Migration In Vitro.

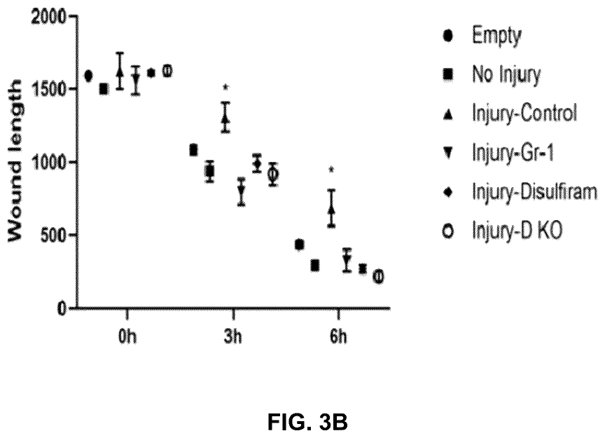

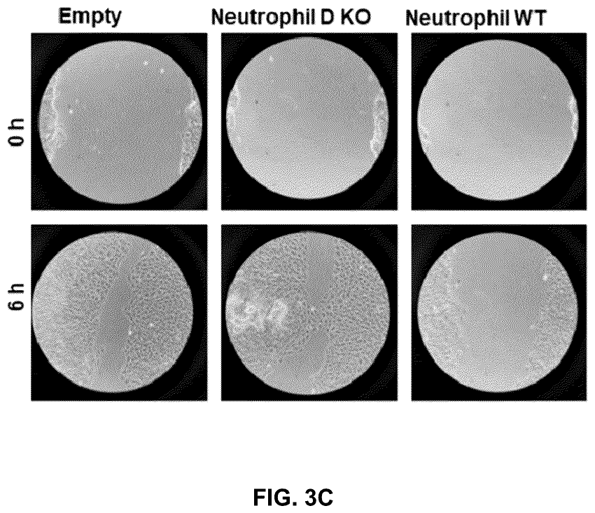

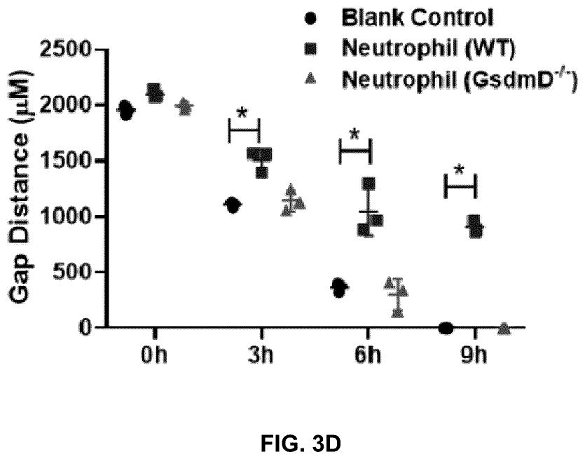

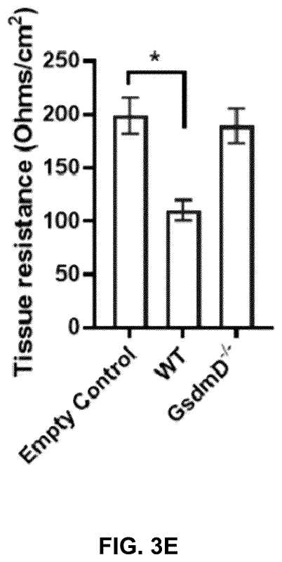

The potential function of neutrophil pyroptosis in corneal wound healing was next tested. Mouse corneas were first dissected with or without injury from WT, GsdmD^{-/-}, Gr-1 treated, and disulfiram treated mice, and cultured the dissected corneas with mCECs. A scratch wound was then created in the mCEC. As shown in the FIG. 3A and 3B, injured WT corneas significantly delayed wound closure of mCECs, as compared to the cells incubated with non-injured WT control corneas. Furthermore, when injured corneas from Gr-1 treated (Gr-1 in FIG. 3A), disulfiram treated (Disulfiram in FIG. 3A), and GsdmD−/− (D KO-Injury in FIG. 3A) mice were used the inhibitory effects on mCECs migration were greatly reduced (FIG. 3A and FIG. 3B). These results suggest that injured corneas secret soluble factors that can suppress mCEC migration.

To test whether these factors were derived from neutrophils, mCECs were co-cultured with isolated primary mouse neutrophils from either WT or GsdmD^{-/-} mice. As shown in the FIG. 3C and FIG. 3D, neutrophils derived from WT mice significantly delayed wound closure of mCECs, as compared to GsdmD^{-/-} neutrophil. Furthermore, TEER measurements indicate that co-culturing mCECs with WT neutrophils can impair mCEC barrier function, as compared to GsdmD^{-/-} neutrophils (FIG. 3E).

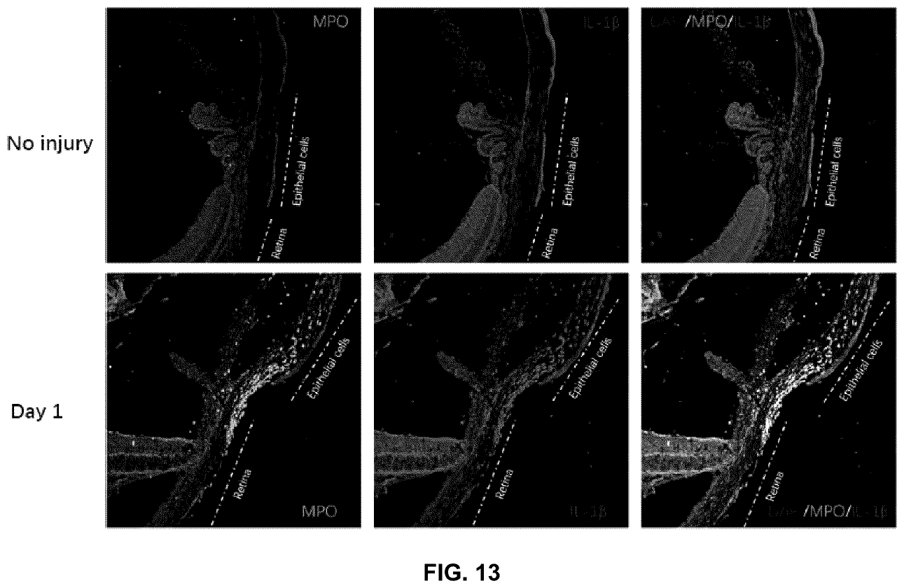

As previously reported, IL-1β and IL-18 are two main cytokines released by immune cells after pyroptosis (Chen, et al. Front Cardiovasc Med. 2022 9:897815; Zhaolin, et al. Cell Prolif. 2019 52(2):e12563). It was hypothesized that IL-1β and IL-18 release from pyroptotic neutrophils suppressed epithelial cell migration. Indeed, IF staining showed increased IL-1β signal after injury and this signal mainly co-localized with MPO in the corneal and limbus (FIG. 13).

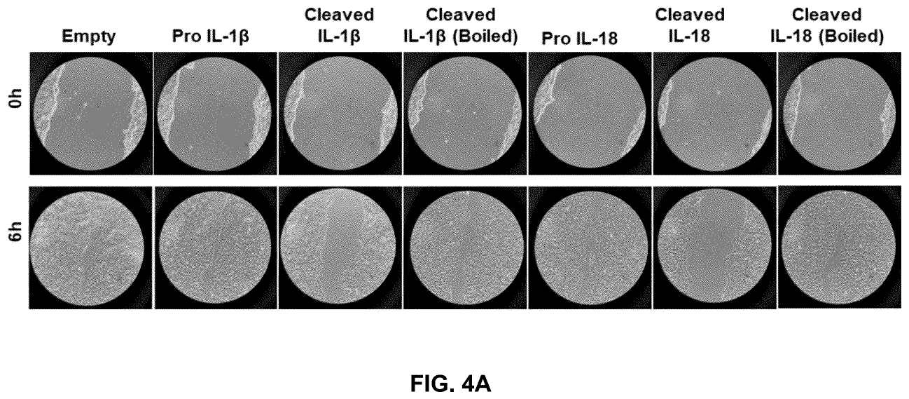

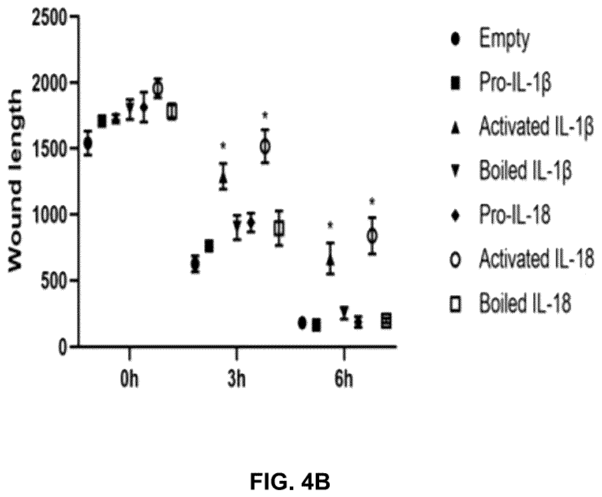

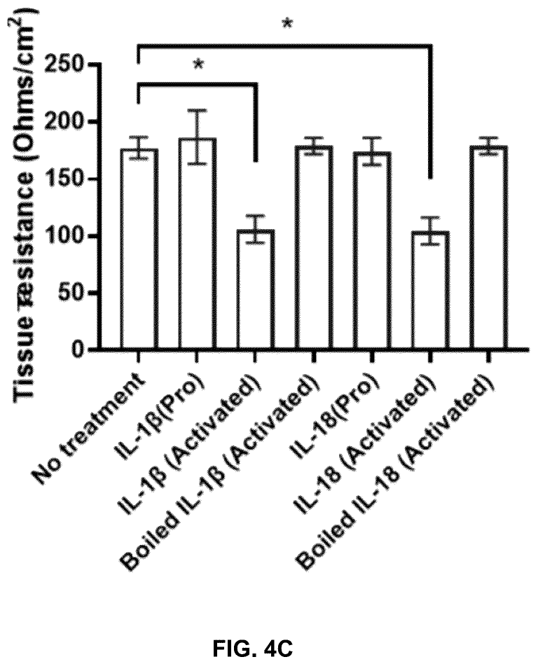

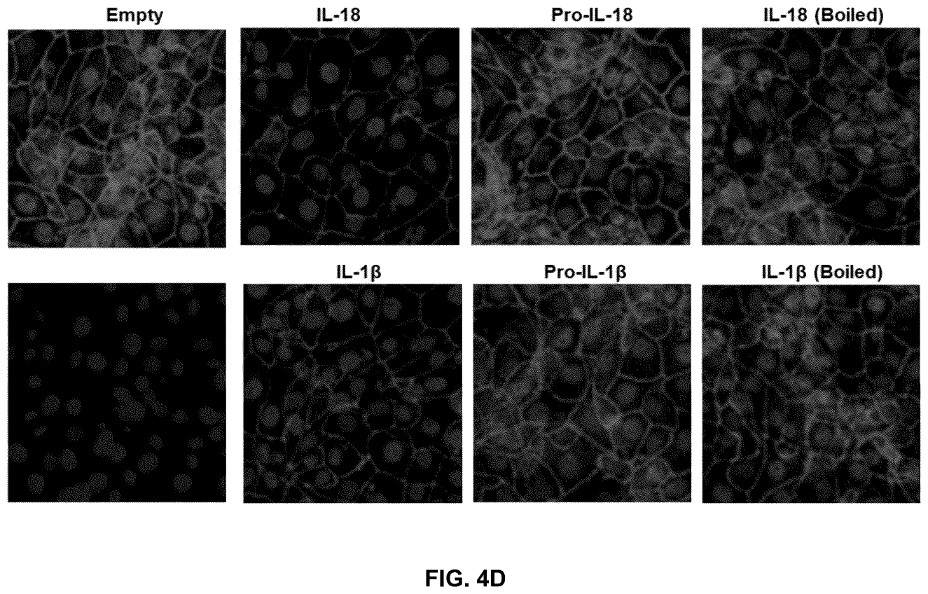

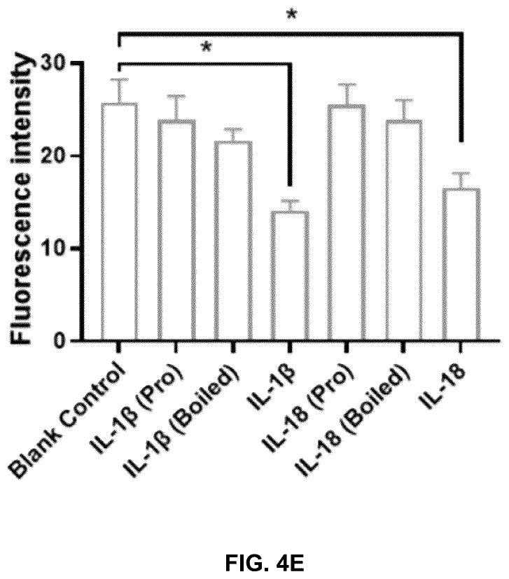

Recombinant pro-IL-1β and pro-IL-18 proteins were generated from E. coli. The proteins were treated with caspase 1 to obtain the active forms. Treatments of active IL-1β and IL-18 significantly delayed mCEC wound closure, while treatments of pro- or boiled IL-1β and IL-18 failed to alter mCEC migration (FIG. 4A and FIG. 4B). Similarly, active IL-1β and IL-18 disrupted epithelial cell tight junctions, as measured by TEER (FIG. 4C) and ZO-1 staining (FIG. 4D and FIG. 4E). Taken together, these results demonstrate that pyroptotic neutrophils release IL-1β and IL-18, which can suppress corneal re-epithelialization after injury.

Faster Re-Epithelialization Prevents Subsequent CNV Through the Wnt5a-sflt1 Signal Axis.

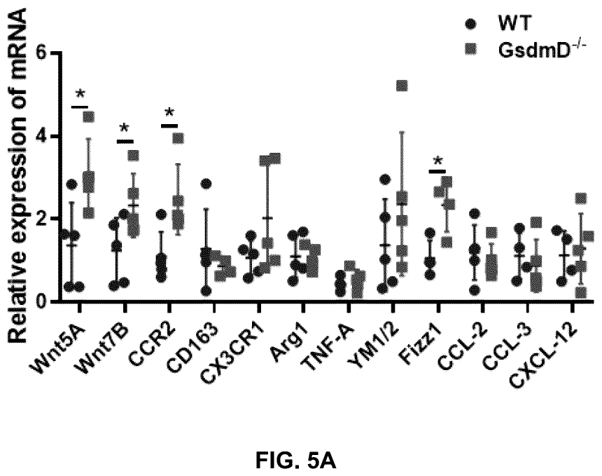

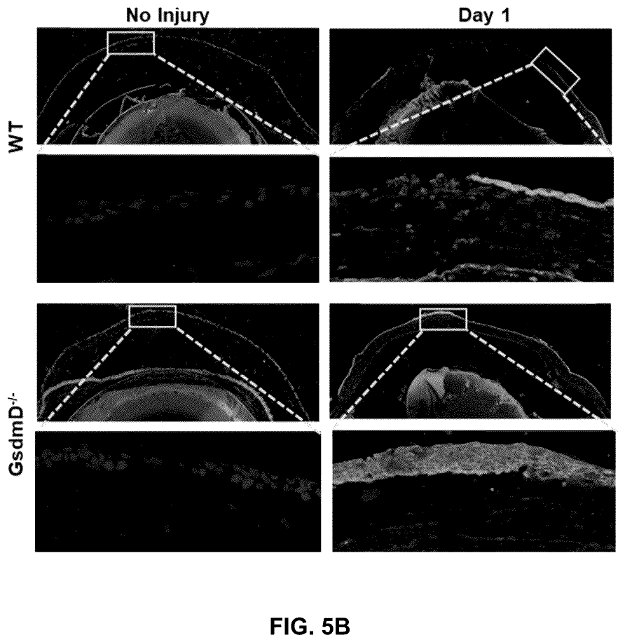

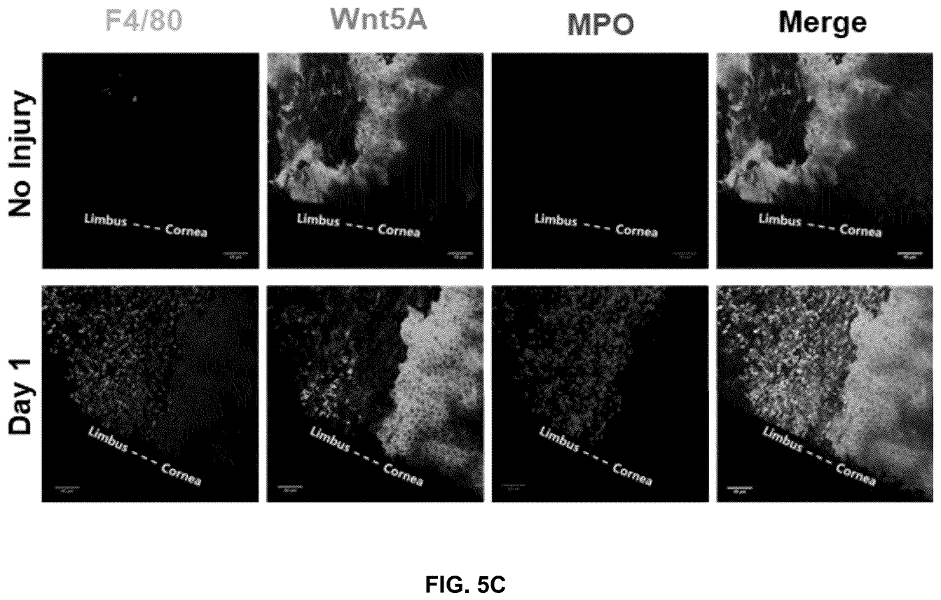

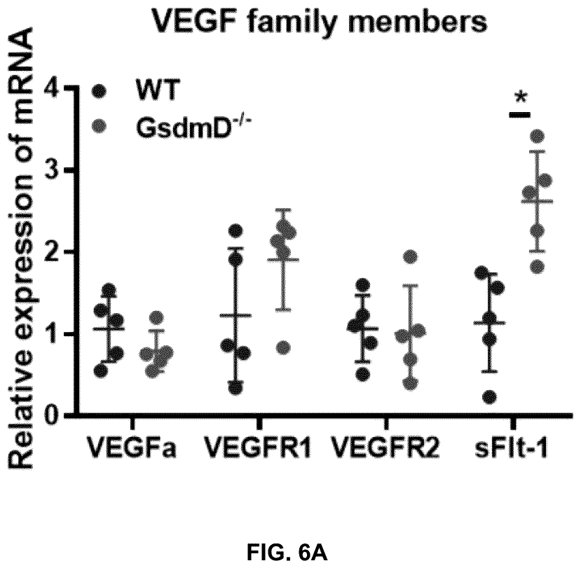

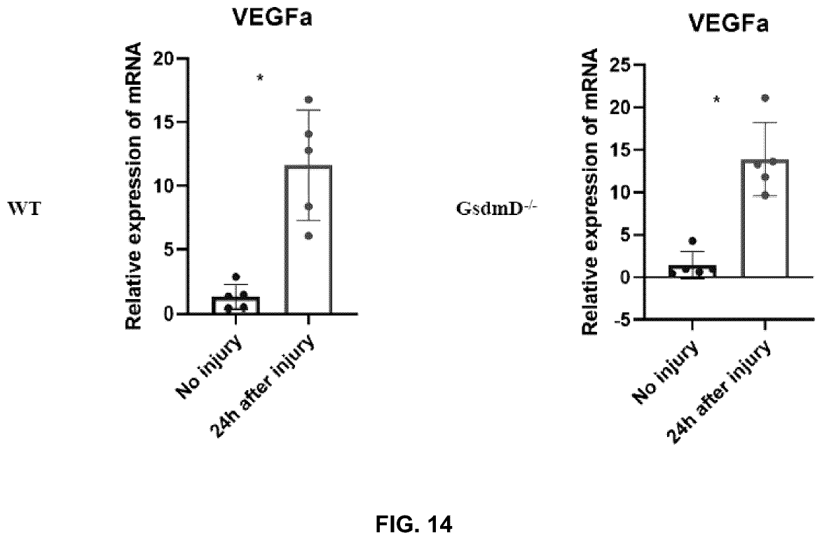

The potential mechanism by which CNV was reduced in GsdmD^{-/-} was next investigated. First tested was whether known angiogenesis factors altered in GsdmD^{-/-} corneas as compared to WT corneas. However, although the mRNA expression level of VEGFa dramatically increased in the cornea after injury in both WT and GsdmD−/− mice, the level of upregulation was similar in both WT and GsdmD^{-/-} mice (FIG. 14). Similarly, VEGFR1 and VEGFR2 levels are similarly upregulated in both WT and GsdmD^{-/-} corneas (FIG. 6A). Thus, other possibilities were also tested. As previous studies have associated macrophages with CNV (Lu, et al. Cornea. 2009 28(5):562-9; Ueta, et al. Proc Natl Acad Sci U S A. 2019 116(47):23705-23713), various macrophage markers were first examined using real-time RT-PCR (FIG. 5A). Expression of Wnt5A, Wnt7B, CCR2, and Fizz1 were significantly increased in GsdmD^{-/-} corneas, as compared to those in WT corneas. Among these molecules, Wnt5A has been associated with inhibiting angiogenesis (Shi, et al. Clin Chim Acta. 2017 471:263-269; Stefater, et al. Blood. 2013 121(13):2574-8; Xu, et al. Inflammation. 2019 42(3):818-825; Murdoch, et al. Biochem Soc Trans. 2014 42(6):1665-70; Stefater, et al. Nature. 2011 474(7352):511-5). To confirm the real-time RT-PCR finding, IF staining of Wnt5A in mouse corneas was performed (FIG. 5B). Surprisingly, strong Wnt5A signal was found in newly generated corneal epithelium after injury but was not detectable in the intact mouse cornea (FIG. 5B). Flat mount staining clearly shows that Wnt5a was mainly expressed in the limbal epithelial compared to the corneal epithelium of the intact ocular surface (FIG. 5C). However, after injury, Wnt5A became highly expressed in the newly differentiated epithelial cells (FIG. 5C). Further, Wnt5A induction was greater in GsdmD^{-/-} corneas than that in WT corneas after injury, which coincided with the significantly faster re-epithelialization rate that we observed in GsdmD^{-/-} corneas.

It is well known that the key molecule maintaining corneal avascularity is soluble VEGF receptor 1 (sflt1) (Stefater, et al. Nature. 2011 474(7352):511-5; Ambati, et al. Nature. 2006 26;443(7114):993). More importantly, previous studies have shown that sflt1 is one of downstream targets of the Wnt5A mediated anti-angiogenic signaling pathway (Karki, et al. Am J Physiol Heart Circ Physiol. 2017 313(1):H200-H206). sflt1 mRNA expression level was next quantified, showing that expression increased in the GsdmD^{-/-} corneas, as compared to that in WT corneas, after injury (FIG. 6A); other angiogenic factors (VEGFa, VEGFR1, VEGFR2) remained similar between the two groups. Consistent with observation of Wnt5A, IF staining results showed that sflt1 was mainly located in the corneal epithelium and sflt1 signal increased after injury (FIG. 6B). Taken together, these studies suggested that the Wnt5A/sflt1 signal axis in newly generated epithelial cells may play an important role in controlling CNV following injury. This would explain why injured GsdmD^{-/-} corneas have faster re-epithelialization and develop less CNV when compared to WT corneas.

Bone marrow transplantation from GsdmD−/− to WT mice or neutrophil depletion accelerates corneal wound healing.

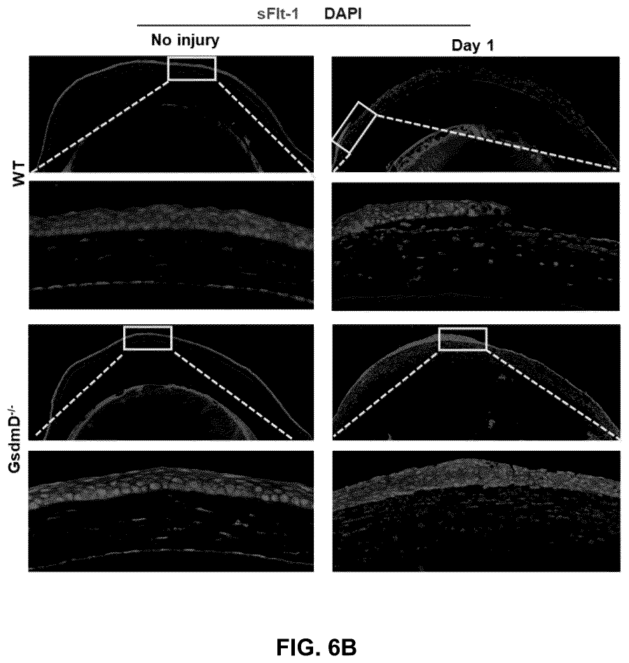

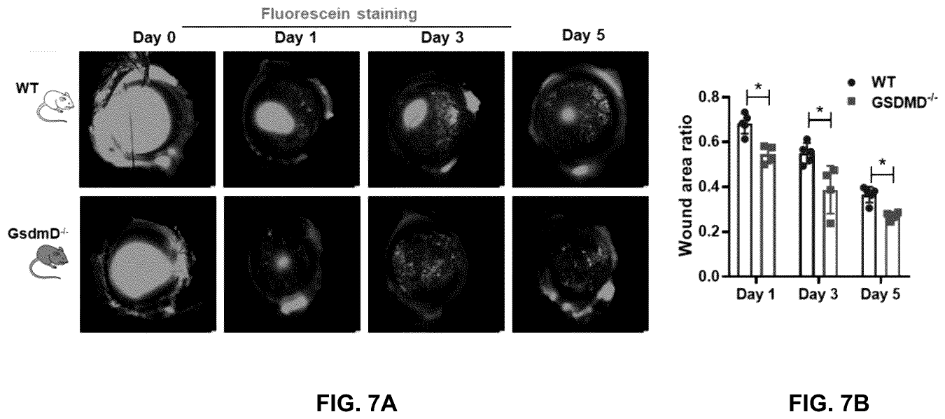

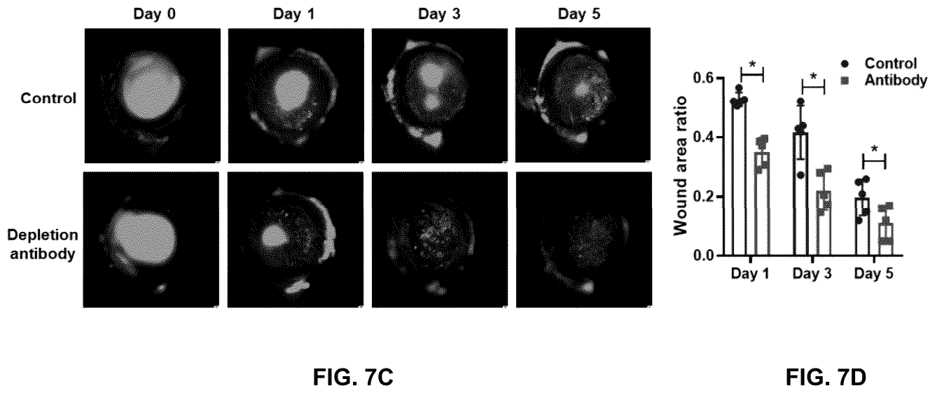

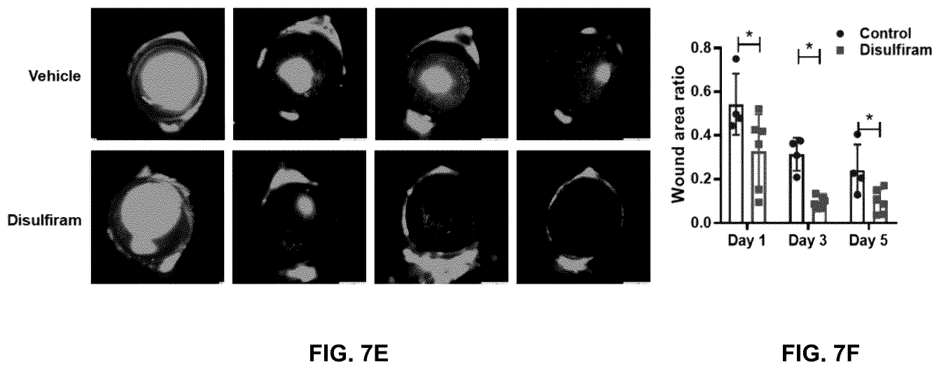

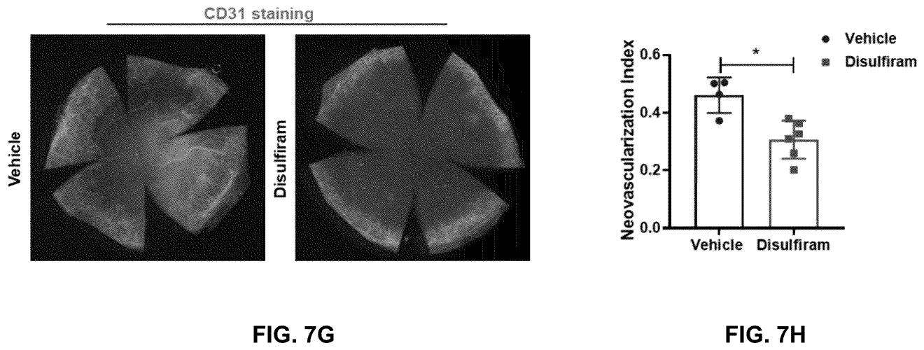

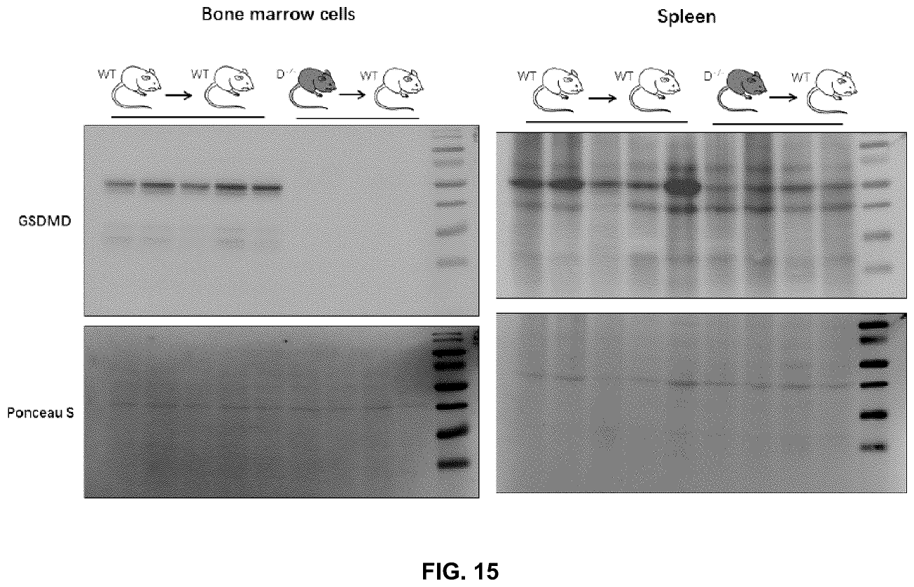

In order to test the contribution of neutrophils in corneal wound healing, a bone marrow transplantation experiment was performed. The following two groups of chimeric mice were generated: WT bone marrow to WT mice (as control), GsdmD^{-/-} bone marrow to WT mice. The efficiency of bone marrow transplantation was confirmed by Western blot analysis of bone marrow and spleens (FIG. 15). Following corneal injury, mice that received GsdmD^{-/-} bone marrow showed significantly quicker wound healing than mice that received WT bone marrow (FIGS. 7A and 7B).

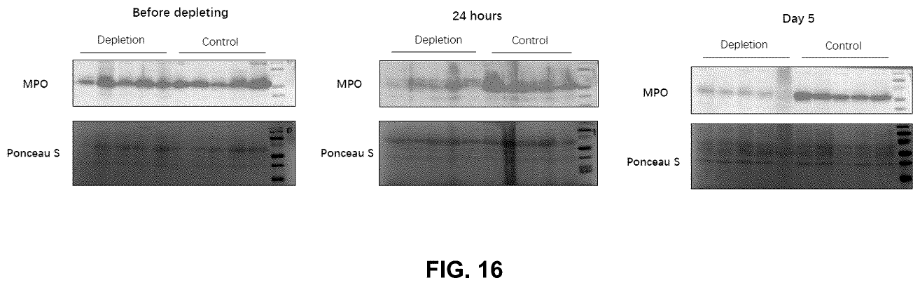

Since bone marrow gives rise to many cell types, including monocytes, macrophages, neutrophils, and others, the bone marrow transplantation experiments can only partially suggest the potential contribution of neutrophils in corneal wound healing. A published double antibody-based depletion strategy (Boivin, et al. Nat Commun. 2020 11(1):2762) was next employed to deplete neutrophils before corneal wounding (FIG. 16). Consistent with the bone marrow transplantation experiments, mice injected with antibodies showed significantly improved re-epithelialization, compared to control animals (FIGS. 7C and 7D). Taken together, these studies suggest that pyroptotic neutrophils inhibit corneal wound healing.

### Pyroptosis Inhibition Promotes Corneal Wound Healing and Reduces CNV.

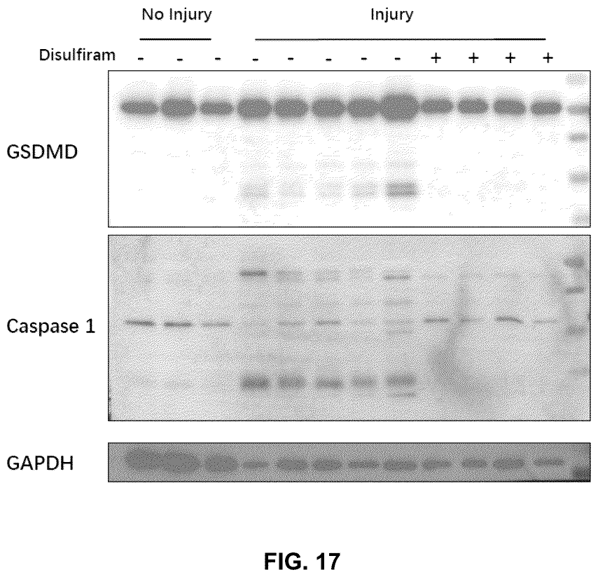

While neutrophil pyroptosis plays a detrimental role during corneal wound healing, the initial neutrophil infiltration is critical for removing dead corneal cells and debris, which is part of the healing process. Thus, complete depletion of neutrophil may not be the ideal option to promote normal corneal healing. Disulfiram is an FDA approved drug, which has been recently been identified as a pyroptosis inhibitor (Hu, et al. Nat Immunol. 2020 21(7):736-745). Thus, tested was whether application of disulfiram could be an effective means to promote corneal wound healing. Indeed, as shown in FIGS. 7E and 7F, the mice treated with disulfiram showed dramatically accelerated wound healing. Furthermore, the IF staining of CD31 showed that CNV was significantly reduced in the mice receiving disulfiram treatment (FIGS. 7G and 7H). To confirm treatment of disulfiram could inhibit post-injury pyroptosis, Western blot analysis was performed on corneal samples before and after injury. As expected, disulfiram treatment effectively inhibited pyroptosis after corneal wounding (FIG. 17). Thus, this study suggests that disulfiram might be an effective means to promote corneal wound healing and inhibit post-injury CNV.

### Discussion

GsdmD has recently been regarded as a negative regulator of innate immunity where GsdmD deficiency has been shown to delay neutrophil death and enhance host response to bacteria (Kambara, et al. Cell Rep. 2018 22(11):2924-2936). The role of neutrophils in the wound healing process is mainly considered beneficial (Ellis, et al. Curr Dermatol Rep. 2018 7(4):350-358). Neutrophil infiltration to the wound area is one of the earliest steps of wound healing, enabling phagocytosis of cellular debris and bacteria, allowing for decontamination of the wound (Wallace, et al. in StatPearls. (StatPearls Publishing Copyright® 2021, StatPearls Publishing LLC., Treasure Island (FL), 2021)). Neutrophils extracellular traps (NET)s have also been considered to have dual functions in wound healing. NETosis, originally only considered a defensive mechanism, is now known to induce detrimental effects on tissue physiology, exacerbating pathologies (Sabbatini, et al. Cells. 2021 10(3):494). As one example, alkali burns induced NET in the cornea impairs epithelium migration (Wan, et al. Front Immunol. 2020 11:551057). Although the literature supports the dual functions of neutrophils in the wound healing process, the role of neutrophil pyroptosis in corneal healing has not been studied.

In the present study, neutrophils infiltrated the injured cornea as early as 3 hrs and peaked at approximately 24 hrs after alkali induced injury. Infiltrating neutrophils quickly undergo pyroptosis. The pyroptotic neutrophils release IL-1β and IL-18 that suppress migration of epithelial cells and compromised epithelial integrity. Consistently, IL-1β has been reported to delay corneal wound healing by impairing migration and inducing apoptosis of epithelial cells (Basso, et al. J Periodontol. 2016 87(8):990-6). Tong Xu et al., also reported that IL-1β increased tight junction permeability in bovine mammary epithelial cells (Xu, et al. J Cell Biochem. 2018 119(11):9028-9041). This study revealed that neutrophils are likely the main source of IL-1β and IL-18, which can mitigate re-epithelialization after corneal wounding.

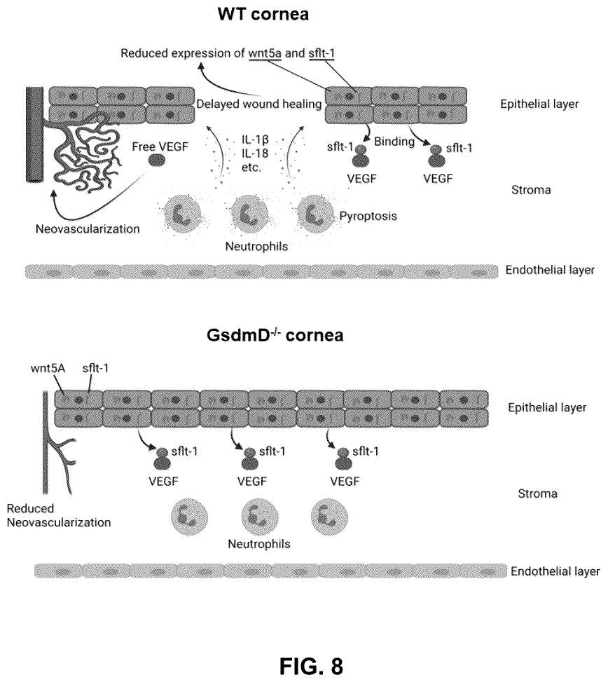

Furthermore, the current study also revealed a potential role for newly generated epithelium in regulating CNV. These data demonstrated that the newly generated corneal epithelial cells express high levels of Wnt5A, which has been reported as an angiogenesis inhibitor by upregulating soluble VEGF receptor 1 (Stefater, et al. Nature. 2011 474(7352):511-5). It is well known that the balance between VEGFa and sflt1 is key to maintaining corneal avascularity (Ambati, et al. Nature. 2006 26;443(7114):993). In our study, we show that the corneal epithelium is the main source of sflt1. After alkali injury, while mRNA expression of VEGFa was increased nearly 10-fold in the injured cornea at 24 hours (FIG. 14), loss of epithelium reduced sflt1 expression, likely resulting in a mismatch compared to VEGFa expression. It is believed that this injury induced imbalance of VEGFa and sflt1 might be a key reason post-injury CNV is observed. Therefore, the rapid repair of the corneal epithelium plays a key role in restoring the balance between VEGFa and sflt1 and reducing subsequent CNV (FIG. 8).

Disulfiram is an FDA approved drug, which has been reported to be a pyroptosis inhibitor (Hu, et al. Nat Immunol. 2020 21(7):736-745). Disulfiram can not only inhibit the release of mature IL-1β, but the drug can also inhibit the cleavage of caspase 1 and GsdmD, needed for pyroptosis activation. Mice treated with disulfiram demonstrated improved healing outcomes compared to controls and as such, disulfiram could be an effective treatment to promote corneal healing and inhibit CNV post-injury.

Taken together, these results reveal the important role of neutrophil pyroptosis during the cornea wound healing process. The inhibition of neutrophil pyroptosis after injury could be a potential clinical therapeutic to promote corneal healing and inhibit CNV.

### Materials and Methods:

### Animals

All animal care and usage followed NIH guidelines and were in accordance with the ARVO Statement for the Use of Animals in Ophthalmic and Vision Research. Rodent studies received IACUC approval by The Ohio State University. For all corneal wound healing models, injury was induced under anesthesia, all animals received topical antibiotics, and topical and systemic analgesics were provided for at least 72 h. To induce corneal injury, a 1.5 mm filter paper soaked in 1N NaOH was applied to the axial cornea for 30 seconds. The filter paper was then removed and the cornea rinsed with PBS for two minutes. After the cornea was thoroughly rinsed, fluorescein stain was applied to the cornea to verify corneal ulceration.

### Preparation of Recombinant IL-1β and IL-18 Proteins

The coding sequences of human pro-IL-18 and pro-IL-1β were cloned into the PET28a vector, where an N-terminal 6xHis tag is added. The purification procedure is similar to the published protocol with minor modifications (Ramirez, et al. J Biol Chem. 2018 293(18): 7058-7067). Briefly, the respective plasmid was transformed into BL21 (DE31) chemically competent cells. A single colony was picked to grow in LB media at 37° C. until OD600 researched 0.6, and 0.5 mM IPTG was used for overnight induction at ˜16° C. Harvested cells were collected by centrifugation, followed by resuspension in 50 mM Hepes (pH 7.5), 5 mM MgCl₂, 1 mM PMSF, ˜0.5 mg/ml lysozyme and 50-100 U DNase I. After sonication, the cell lysates were centrifuged. The supernatant was then applied to a Ni-NTA affinity chromatography, where buffer A contained 50 mM Hepes (pH 7.5) and Buffer B contained an extra 500 mM imidazole. Fractions with the highest purities were pooled and applied to a diethylaminoethyl-cellulos (DEAE) ion exchange chromatography, where buffer A′ contained 50 mM Hepes (pH 7.5) and Buffer B′ contained an extra 500 mM NaCl. As judged by SDS PAGE, fractions with >95% purity were pooled and dialyzed in 50 mM Hepes (pH 7.5) and 100 mM NaCl.

Recombinant human caspase-1 was purchased from Enzo Life Sciences (ALX-201-056). The cleavage of pro-IL-18 or pro-IL-1β was performed at 37° C. in PBS buffer containing 2 mM DTT.

### Drug or Antibody Treatment

For disulfiram treatment of mice, disulfiram was dissolved in sesame oil (12.5 mg/ml). Mice were injected 16 hours before injury through intraperitoneal injection (50 mg/kg); injection continued daily for the duration of the experiment. For Gr-1 antibody treatment, mice were injected 16 hours and immediately before injury through intraperitoneal injection (100 μg/mouse).

### Bone Marrow Chimeras

Mice were subjected to split-dose irradiation, first with a 350 rad dose, followed by a 950 rad dose 24 hours later. Four hours after the second dose of irradiation, mice were injected intravenously with flushed bone marrow cells from the femur of donor mice. The following two groups of chimeric mice were generated: WT to WT, Gsdmd^{-/-} to WT. The mice were underwent corneal injury 6 weeks after bone marrow transfer and the reconstitution efficiency were determined by Western blot after sacrificing the mice.

### Cell Lines and Treatments

Mouse primary corneal epithelial cells (mCEC)s were purchased from Cell Biologics, Inc. (Chicago, IL). mCECs were maintained in mouse epithelial cell medium (Cell Biologics, Inc., cat.no. M6621). THP-1 cells were maintained in Dulbecco's modified Eagle's medium (DMEM) supplemented with 10% fetal bovine serum and 1% penicillin/streptomycin in 5% CO₂ incubator.

THP-1 cells were first differentiated by incubation with 0.2 μg/ml PMA for 36 to 48 hours and then starved without FBS for 24 hours and then primed with LPS (1 μg/ml) for 3 hours before treatment with nigericin (20 μM). For disulfiram treatment, cells were treated with 40 uM disulfiram (diluted in DMSO, 12.5 mg/ml) for 1 hour before treatment with nigericin.

### Cell Scratch Wound Healing Assay

To induce cellular migration, an in vitro scratch test was performed using mCECs. Cells were treated with dissected corneas or cytokines when they reached confluence, were subsequently scratched with a micropipette tip, and incubated for up to 12 hrs. Images were taken at 0, 3, 6, and 9 hours after the initial scratch. Images of wound closure were quantified by using ImageJ software.

For co-culture experiments, mCECs were cultured in 24 well plates. Upon reaching confluence, the primary neutrophils were isolated and immediately seeded into Transwell inserts (0.4 μm polyester membrane). The mCEC subsequently underwent scratch formation using a micropipette tip; image acquisition and analysis were performed as described above.

### Transepithelial Electrical Resistance (TEER) Measurement

mCEC were seeded in transwell inserts (2×10⁵ per well) and allowed to become confluent. For the protein treatment, proteins were added into the culture medium 16 or 4 hours before measurement; the EVOM2 instrument was used to measure the TEER. For the co-culture experiments, mCEC were seeded in the transwell inserts and allowed to become confluent. The isolated primary neutrophils were then seeded into 24 well plates and the EVOM2 instrument was used to measure the TEER at 24 hr.

### Mouse Primary Neutrophil Isolation

Bone marrow samples were obtained as previously described. Briefly, lower limbs were collected from euthanized mice. All attached soft tissue was removed to fully expose the femurs and tibias. An 18G needle was inserted into hollow of the bone and pulse flush buffer (2% FBS in PBS) used to flush marrow into a 15 ml sterile centrifuge tube. The solution was then filtered with a 70-100 μm filter. The solution was centrifuged at 10,000 ×g, and the bone marrow cells were resuspended in red blood cell lysis buffer for 5 minutes.

Separation of neutrophils using density gradient centrifugation was performed as previously described (Swamydas, et al. J Vis Exp. 2013 (77):e50586). The bone marrow cells were resuspended in 1 ml of ice-cold sterile PBS. Using a 15 mL conical tube, 3 ml of Histopaque 1119 was first added followed by an overlay of 3 mL of Histopaque 1077. The bone marrow cell suspension was then overlaid on the Histopaque 1077. The samples were centrifuged for 30 min at 2,000 rpm at 25° C. Neutrophils were then at the interface of the Histopaque 1119 and Histopaque 1077 layers. The collected neutrophil were washed twice with culture medium and centrifuged at 1,000 g for 3 min.

### Western Blot

Protein lysates from indicated tissue and cell sources were separated by SDS-PAGE. The antibodies used in this study were as follows: anti-GsdmD antibody (Abcam, Cat. No. ab219800); anti-IL-1β antibody (Abcam, Cat. No. ab4722); anti-human caspase 1 antibody (Cell Signaling Tech, Cat. No. #3866); anti-mouse caspase 1 antibody (BioLegend, Cat. No. 645102); anti-MPO antibody (R&D systems, Cat. No. AF3667); anti-GAPDH antibody (); anti-NLRP3 antibody (Cell Signaling Tech, Cat. No. 15101S). Secondary antibodies, anti-mouse, anti-rabbit, anti-rat, or anti-donkey IgG HRP conjugated, were applied at 1:5000 dilution.

### Quantitative RT-PCR Analysis

Total RNA was extracted from dissected corneas by using TRIzol reagent (Invitrogen, CA, Cat. No. 15596026). 500 ug of total RNA was reverse transcribed by cDNA synthesis (Thermo Scientific, Cat. No. 1651) and the products were subjected to quantitative real-time PCR, carried out by SYBR Green Real-Time PCR mix (Thermo Scientific, Cat. No. A25778).

### Histopathology and Immunofluorescent Staining

Dissected eye from mice were fixed in 4% PFA for 24 to 48 hours at 4° C. The eyes were processed as follows: 2 hours 50% ethanol, 1.5 hours 70% ethanol, 1 hour 80% ethanol, 1 hour 90% ethanol, 30 minutes 95% ethanol, 30 minutes 95% ethanol, 15 minutes 100% ethanol, 15 minutes 100% ethanol, 15 minutes xylene, 15 minutes xylene, 30 minutes Paraffin wax, 1 hour Paraffin wax, 1 hour Paraffin wax. After embedding, five um thick paraffin sections were cut and stained with Hematoxylin-Eosin (H&E) or immunofluorescent staining. For flat mount staining, eye were fixed in 4% PFA at 4° C. for 24 to 48 hours, then transferred to PBS. Corneas were dissected, washed with PBS for 3 times (10 mins for each wash), blocked in blocking buffer (3% BSA and 0.5% Triton X-100 in PBS) for 2 hours at RT, before application of primary antibody (1:100 diluted in blocking buffer) and incubation at 4° C. overnight. Subsequently, corneas were washed 6 times with washing buffer (0.5% Triton X-100 in PBS) for 1 hour each time at RT, and secondary antibodies (1:500 diluted in blocking buffer) applied and incubated at 4° C. overnight. Finally, the corneas were washed with PBS three times at RT for 1 hour each time; four cuts were made in the corneas were to facilitate mounting (DAPI Fluoromount-G, SouthernBiotech, Cat. No. 0100-20) on slides.

The primary antibodies used for IF staining in the present study were anti-Cleaved GsdmD (N-terminal) antibody (Cell Signaling Tech, Cat. No. #50928); anti-IL-1β antibody (Abcam, Cat. No. ab4722); anti-MPO antibody (R&D systems, Cat. No. AF3667); FITC anti F4/80 antibody (Biolegned, Cat. No. 123108); anti VEGF Receptor-1 (Soluble) sflt-1 antibody (Invitrogen, Cat. No. 36-1100); anti Wnt5A antibody (Thermo Scientific, Cat. No. PA5117496); anti-CD31 (BD Biosciences, Cat. No. 550274). Secondary antibodies, Alexa Fluor 546 Donkey anti Rabbit IgG (Life Technologies, Cat. No. A10040), Alexa Fluor 647 Donkey anti Goat IgG (Life Technologies, Cat. No.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating a chemical burn of a cornea in a subject, comprising administering to the cornea of the subject a therapeutically effective amount of composition comprising a pyroptosis inhibitor in a pharmaceutical carrier.

2. The method of claim 1, wherein the pyroptosis inhibitor is compound having the structure of Formula (II), or a pharmaceutically acceptable salt, solvate, or prodrug thereof: 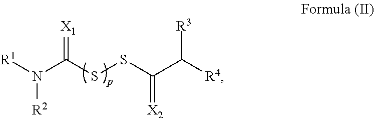
wherein,
X₁ and X₂ are each independently O or S;
p is 1 or 2; and
R¹, R², R³, and R⁴ are each independently H, C₁-C₆ alkyl, C₁-C₆ haloalkyl, C₂-C₆ alkenyl, C₂-C₆ alkynyl, C₃-C₈ cycloalkyl, 4-10 membered heterocycloalkyl, C(O)OR^{1a}, or C(O)NR^{1a}R^{1b}; wherein each is optionally substituted with 1, 2, or 3 R⁵; or R¹ and R² together with the N atom to which they are attached from a 4-10 membered ring, which is optionally substituted with 1, 2, 3, or 4 R⁶; or R³ and R⁴ together with the N atom to which they are attached from a 4-10 membered ring, which is optionally substituted with 1, 2, 3, or 4 R⁷; each R⁵, R⁶, and R⁷ is independently selected from halogen, C₁-C₆ alkyl, C1-C₆ haloalkyl, CN, NO₂, OR^{1a}, C(O)R^{1b}, C(O)NR^{1a}R^{1b}, C(O)OR^{1a}, NR^{1a}R^{1b}, S(O)2R^{1b} and S(O)₂NR^{1a}R^{1b}; each R^{1a} and R^{1b} is independently selected from H, C₁-C₆ alkyl, C₁-C₆ alkenyl, C₁-C₆ alkynyl, cycloalkyl, heterocycloalkyl, aryl or heteroaryl.

3. The method of claim 2, wherein the pyroptosis inhibitor is disulfiram or a pharmaceutically acceptable salt, solvate, or prodrug thereof.

4. The method of claim 1, wherein the chemical burn comprises an alkali burn.

5. The method of claim 1, wherein the chemical burn comprises a vesicant.
